(19) United States
(12) Patent Application Publication
(10) Pub. No.: US 2010/0278687 A1
(43) Pub. Date: Nov. 4, 2010

(54) REMEDIATION OF GYPSUM BOARD USING GASEOUS CHLORINE DIOXIDE

(75) Inventor: John Y. MASON, Odessa, TX (US)

Correspondence Address: HESLIN ROTHENBERG FARLEY & MESITI PC, 5 COLUMBIA CIRCLE, ALBANY, NY, 12203, US

(73) Assignee: SABRE INTELLECTUAL PROPERTY HOLDINGS COMPANY, LLC., Slingerlands, NY (US)

(21) Appl. No.: 12/769,448

(22) Filed: Apr. 28, 2010

Related U.S. Application Data
(60) Provisional application No. 61/173,844, filed on Apr. 29, 2009.
(60) Provisional application No. 61/252,422, filed on Oct. 16, 2009.

(51) Int. Cl. A61L 9/015

(52) U.S. Cl. 422/5

(57) ABSTRACT

In a method for eliminating contaminants in gypsum wallboard that cause noxious sulfide odors, at least one gypsum wallboard surface within an enclosed volume is exposed to chlorine dioxide gas, wherein the chlorine dioxide gas is introduced into the enclosed volume under specified conditions of chlorine dioxide gas concentration and contact time that eliminate the noxious odor-causing contaminants, sulfate reducing or thiophilic bacteria in particular, contained in the gypsum wallboard.

### CROSS-REFERENCE TO RELATED APPLICATIONS

This application claims the benefit of priority from U.S. Provisional Application No. 61/173,844, filed Apr. 29, 2009, and U.S. Provisional Application No. 61/252,422, filed Oct. 16, 2009, the disclosures of which are incorporated herein by reference.

This application is also related to application Ser. No. ______, filed herewith, DECONTAMINATION OF ENCLOSED SPACE USING GASEOUS CHLORINE DIOXIDE, the disclosure of which is incorporated herein by reference.

### FIELD OF THE INVENTION

The present invention relates to the use of gaseous chlorine dioxide for in situ remediation of gypsum board in an enclosed volume to eliminate sulfate reducing bacteria and oxidize reactive metal sulfides in contact with the wallboard.

### BACKGROUND

Gypsum board, also known as wallboard, plasterboard, sheetrock or drywall, consists of wide, flat boards, and is a common building material used in both residential and commercial construction. It is used in a number of applications including interior walls, partitions and ceiling construction. Gypsum board is popular in residential and commercial construction because it is fire resistant, generally inexpensive and, usually, plentiful.

A gypsum board panel consists of an inner core made primarily from wet gypsum plaster, the semi-hydrous form of calcium sulfate (CaSO₄·½H₂O), wrapped on both sides with a fibrous material, typically heavy paper or fiberglass mats, and then kiln dried. The gypsum can either be mined or obtained from flue gas desulphurization.

Usually, drywall used in the United States for residential and commercial construction is manufactured in the United States. However, a shortage during the housing boom in 2005-2007 prompted many builders to buy drywall from China. Beginning in 2008, there have been increasing number of complaints about drywall that is causing a putrid, “rotten-egg” smell in many homes. The most common problem caused by the rotten-egg—sulfur smelling Chinese wallboard is the corrosion of air conditioning equipment, electrical equipment, and other metal fixtures or wiring, which is turning black due to the formation of copper sulfide. Air conditioning units have had a high rate of failure. Homeowners also have complained about respiratory problems they believe are connected to the drywall. Some residents have been forced to move from their homes, and some builders have begun gutting homes and replacing the drywall. Many homeowners have spent thousands to repair or replace wiring, air conditioning units and other systems destroyed by the fumes.

The problems associated with the Chinese drywall appear to increase with high relative humidity or temperature. As a result of these problems and health risks, it is necessary either to replace the contaminated wallboard, which can be very costly, or treat the wallboard and metallic surfaces in situ. There remains a need, however, for an effective means of remediating in situ the source of the corrosion emitting gases, particularly in large structures, and eliminating contamination in the wallboard. Currently, there is no effective means for in situ remediation of Chinese wallboard on a small or large scale.

### SUMMARY OF THE INVENTION

The present invention relates to a method for eliminating off-gassing of reduced sulfur and sulfur gases from gypsum wallboard that comprises exposing at least one gypsum wallboard surface to chlorine dioxide gas.

The present invention also relates to a method for in situ remediation of gypsum wallboard that comprises exposing at least one gypsum wallboard surface to about 9000 ppmᵥ hours of chlorine dioxide gas.

The present invention further relates to a method for eliminating contaminants in gypsum wallboard that cause noxious sulfide odors, the method comprising: exposing to chlorine dioxide gas at least one gypsum wallboard surface within an enclosed volume, introducing the chlorine dioxide gas into the enclosed volume under conditions of chlorine dioxide gas concentration, contact time, and humidity effective to eliminate the noxious odor-causing contaminants in the gypsum wallboard.

### DETAILED DESCRIPTION

The method of the present invention provides for the elimination of contaminants that cause noxious sulfur odors in gypsum wallboard. “Elimination of contaminants” is defined as eliminating at least 95% of contaminants, or preferably eliminating at least 98% of contaminants, or more preferably eliminating at least 99% of contaminants. As it pertains to “off-gassing”, “eliminate” means to reduce the level of sulfur-containing volatiles to a level that is not detectable by human smell. For example, in its toxological profile for hydrogen sulfide, the Agency for Toxic Substances & Disease Registry identifies an odor threshold of 0.3 ppm.

Preferably, the enclosed volume is at a temperature of about 10° C. (50° F.) to about 32° C. (90° F.), more preferably about 18° C. (65° F.) to about 29° C. (85° F.). Chlorine dioxide gas is introduced into the enclosed volume at a concentration of about 25 ppmᵥ to about 10,000 ppmᵥ, preferably about 500 ppmᵥ to about 5,000 ppmᵥ, and at a CT value of about 150 ppmᵥ-hrs to 50,000 ppmᵥ-hrs, preferably about 1000 ppmᵥ-hrs to 29,000 ppmᵥ-hrs

In certain embodiments of the invention, the contaminants in the gypsum wall board comprise bacteria, in particular, sulfate reducing or thiophilic bacteria. The enclosed volume may further include objects selected from the group consisting of metallic objects, non-metallic objects, and combinations thereof.

Metallic objects within the enclosed volume are formed from metals selected from the group consisting of steel, aluminum, iron, copper, chromium, lead, and combinations thereof. Non-metallic objects are formed from materials selected from the group consisting of wood, brick, stone, cinder concrete, ceramic tile, ceiling tile, carpet, woven fabric, and combinations thereof.

In one embodiment of the invention, the method further comprises exposing at least one gypsum wallboard surface to about 9000 ppmᵥ hours of chlorine dioxide gas. In another embodiment, the chlorine dioxide gas is introduced into the enclosed volume at a CT value of chlorine dioxide equal toy (ppmᵥ/hrs), wherein y=6x²−870x+32100±1000, x being equal to % RH. In a further embodiment, the chlorine dioxide gas is introduced into the enclosed volume at a CT value of about 150 ppmᵥ-hrs to about 50,000 ppmᵥ-hrs preferably about 1000 ppmᵥ-hrs to about 29,000 ppmᵥ-hrs.

As set forth in more detail below, the applicant has identified the presence of hydrogen sulfide (H₂S) in the wallboard as the cause of the rotten-egg smell. Initial testing has shown growth of sulfate-reducing bacteria (SRBs) and other sulfur bacteria on samples of Chinese wallboard from different structures.

Under anaerobic, ambient conditions, sulfate-reducing bacteria (SRB) produce hydrogen sulfide gas and other reduced sulfur gases via the reduction of sulfur compounds, such as sulfate or elemental sulfur. A by-product of SRB, hydrogen sulfide is a clear, colorless and highly corrosive gas. At low concentrations, hydrogen sulfide is an irritant in that it smells like rotten eggs. At higher concentrations, it irritates the eyes, nose and respiratory. At very high concentrations, it can be extremely dangerous and deadly.

Gypsum board is susceptible to moisture accumulation, thereby permitting the growth of bacteria within the wallboard. In addition to corrosion, the presence of bacteria and subsequent release of hydrogen sulfide and other reduced sulfur gases from wallboards may also cause numerous deleterious health effects, including allergic reactions and respiratory problems.

The present invention provides a method for in situ remediation of gypsum wallboard comprising exposing said contaminated gypsum wallboard to chlorine dioxide gas. It has been determined that chlorine dioxide fumigation of an enclosed structure at about 9000 ppmᵥ hours (at 65% RH and 65° F.) will result in eliminating the growth of SRBs and sulfur bacteria and will oxidize reactive sulfides causing corrosion. As discovered by the applicant, the chlorine dioxide gas diffuses through the Chinese wallboard, which after being exposed to the requisite concentration and contact time, provides for the elimination of the SRBs and oxidation of any reactive sulfides causing corrosion. This vitiates the need to rip out and replace the contaminated wallboard. It is an aspect of this invention to mitigate microbially offgas-induced corrosion of structural elements comprise of copper or iron, within the walls of a structure by exposing at least one surface of the gypsum wallboard to chlorine dioxide gas.

### Example 1

In one embodiment, an outline for testing the Chinese wallboard is as follows:

1. Collection of Samples—Two structures are identified within the affected area that has exhibited effects associated with gas evolution from wallboard. The structures are investigated and any effects such as discoloration, corrosion, and presence of odors noted. Drawings are prepared of the structure with the areas of effect noted, photographed and documented. Samples of discolored metal and or materials are collected for analysis, and samples of the drywall are collected for testing.

2. Determination of Presence of SRBs in Wallboard Samples—Two samples of domestic wallboard and two samples of Chinese Wallboard from different structures are tested to determine the presence of sulfate reducing bacteria. A 2 mm core is taken at least 4 inches from the edge of the wallboard and homogenized in 10 ml of an anaerobic SRB broth. 1 ml of the broth is transferred to 10 ml of broth in an anaerobic container and diluted serially out to a 104th dilution. The broth bottles are incubated at 25° C. for 21 days. Each day growth will be recorded. A lab blank negative control is performed on the initial broth.

3. Determination of BOD and in Gypsum Wallboard Core—Two samples of domestic wallboard and two samples of Chinese wallboard from different structures are tested to determine the presence and level of biochemical oxygen demand (BOD) within the gypsum board. The paper is removed from the samples and the core material is extracted. 10 grams of the core material is crushed and mixed with 90 grams of distilled water. The sample is allowed to stand for 24 hours and then filtered through a 0.45-micron filter. The filtrate is tested by standard method 5210B for the determination of BOD.

4. Determination of Volatile Solids in Gypsum Wallboard Core—Two samples of domestic wallboard and two samples of Chinese wallboard from different structures are tested to determine the percent volatile solids. A core sample of each test subject is isolated as in step 2 and tested for total and fixed volatile solids by AWWA standard methods 2450-E.

5. Determination of Soluble Sulfate and Sulfite (Free) in Gypsum Wallboard Core

Two samples of domestic wallboard and two samples of Chinese wallboard from different structures are tested to determine the presence and level of soluble or free sulfate within the core material. 10 grams of the core material from each sample is crushed and mixed with 90 grams of distilled water and allowed to soak for 24 hours. The samples are filtered and the filtrate analyzed for sulfate content by ASTM D516-07 and for sulfite by ASTM D 1339-84.

6. Determination of Evolution Potential for H₂S Gas—Samples collected that show discoloration and samples from five sections of wallboard are analyzed for the potential to evolve hydrogen sulfide gas using a Garret Gas Train. Two 10 gram samples of each material are tested. One sample is tested at ambient pH to determine the free available hydrogen sulfide that can be evolved. The second sample is tested at a pH of 2 to determine the total available hydrogen sulfide. This procedure is carried out at temperatures of 65, 75, and 85° F.

The next step comprises producing chlorine dioxide gas by using an apparatus such as a chlorine dioxide generator, e.g., as disclosed and claimed in U.S. Pat. No. 6,468,479, the disclosure of which is incorporated herein by reference. The chlorine dioxide is generated either directly as a gas or, more preferably, as an aqueous (or other suitable liquid carrier) solution of chlorine dioxide. The generator is preferably run using an excess of sodium chlorite to reduce the possibility of generating chlorine gas as an impurity. The carrier liquid in the generator is preferably water. In an aqueous solution, chlorine dioxide solution equilibrium partial pressure is optimally kept below about 26,000 ppmᵥ (corrected for standard temperature and pressure).

If the space to be remediated contains materials that are potentially susceptible to corrosion, the chlorine dioxide should be of the highest possible purity. Specifically, chlorine gas should be present in the introduced chlorine dioxide gas at a level less than about 5%, preferably less than about 0.5%. Several chemical means of generating chlorine dioxide and their corresponding chlorine dioxide precursor chemicals are known in the art, and the choice of suitable means and chemicals is within the abilities of the skilled artisan. For example, other exemplary chemical means of generating chlorine dioxide are disclosed in U.S. Pat. Nos. 4,689,169 (Mason et al.), 5,204,081 (Mason et al.), 5,227,306 (Eltomi et al.), 5,258,171 (Eltomi et al.), 5,965,004 (Cowley et al.), and 6,645,457 (Mason et al.), the disclosures of which are hereby incorporated by reference.

The method comprises the further steps of introducing the chlorine dioxide gas into the volume requiring remediation, distributing the introduced chlorine dioxide gas in said volume, and maintaining the chlorine dioxide gas within said volume at a concentration and for a sufficient duration of time to permit gaseous penetration of included contents, as described in U.S. patent application Ser. Nos. 11/270,973 and 11/576,498, the disclosures of which are incorporated herein by reference.

In particular, the generated chlorine dioxide is transferred directly, or alternatively, indirectly via a storage tank, to a high gas:liquid ratio emitter. In one preferred embodiment, the emitter is an apparatus such as a gas/liquid contactor having a high efficiency mist eliminator and very low liquid/gas rates. In one embodiment, the emitter is an apparatus such as a stripper.

The emitter is operated to maintain the gaseous chlorine dioxide concentration substantially below the explosion limit of chlorine dioxide in the air. Prior to generation of the chlorine dioxide, the emitters may be used with water alone to raise the relative humidity in the volume requiring remediation, with adjustment of the temperature. Alternatively, the humidification and remediation can be done simultaneously using the same apparatus by the appropriate adjustment in the temperature of chlorine dioxide solution. This pre-humidification may be helpful in swelling the spore coats of resistant molds and may aid in remediating particularly recalcitrant species. Control of humidity level during remediation may also aid in gaseous penetration of some porous surfaces.

The treatment is conducted in reduced illumination, preferably substantially dark, to minimize the decomposition of chlorine dioxide to chlorine. The process is monitored with the use of an infrared camera or similar device.

Next, the variable generation rate of chlorine dioxide gas is initiated. The initial rate is high to provide sufficient chlorine dioxide to penetrate the various surfaces demands within the volume requiring remediation. This rate is predetermined to accommodate the surface demand as well as to provide the initial charge of the volume requiring remediation to a predetermined chlorine dioxide residual level. The chlorine dioxide generation rate is then reduced appropriately to maintain the predetermined chlorine dioxide concentration in the air of the volume requiring remediation for a predetermined time. This can be achieved by a number of means, such as lowering the concentration of chlorine dioxide in the solution that is fed to the emitter, or lowering the flow rate of the chlorine dioxide solution to the emitter.

The chlorine dioxide gas concentration is determined to compensate for the decay or loss rate from the volume requiring remediation. The volume requiring remediation is preferably to be at slightly negative pressure to areas outside of it and efforts are made to seal off the volume through the use of strippable sealant, such as foam that sets up hard. In addition, the volume to be remediated can be enclosed within a substantially light impervious tent while undergoing remediation so as to avoid light-induced degradation of the introduced chlorine dioxide gas. In another embodiment, the tent is substantially impervious to gas.

Once the required time weighted average concentration and contact time are attained, then the generation of chlorine dioxide is stopped.

### Example 2

In another embodiment, the SRB kill in the Chinese wallboard can be determined as follows:

Two square (100 cm×100 cm) samples of ⅝ inch Chinese wall board are collected from the structure that reported odor issues from different areas of the structure. If possible, the samples are collected near areas that exhibit discoloration of copper of metal. The samples are split into four 25 cm×25 cm squares with the exposed ends of the sheetrock taped and painted with the same paint used within the structure. Two squares from each sample are placed into a chlorine dioxide fumigation chamber and fumigated with chlorine dioxide to a CT value of 9000 ppmᵥ hours at 65% RH and 65° F. The other two squares from each sample are held in sealed bags as test blanks.

Upon completion of the test, each of the samples can be sampled as follows: Each core sample is homogenized in 10 ml of an anaerobic SRB broth, and 1 ml of the broth is transferred to 10 ml of broth in an anaerobic container and diluted serially out to a 104th dilution. The broth bottles are incubated at 25° C. for 21 days. Each day growth is recorded, and a lab blank negative control is performed on the initial broth.

In the next step, the generator, storage and emitter are purged with fresh water. Once this is complete, the water may be injected with an alkalizing and dechlorinating agent or other functional chemistry (e.g., ascorbic acid) that will scrub the chlorine dioxide. This scrubbing solution is then fed to the emitter and with the blowers still in operation, the emitter begins to scrub chlorine dioxide out of the environmental air composition within the said volume that has been remediated. This process is continued until the environmental air composition within the volume that has been remediated is returned to acceptable limits for reopening to the exterior environment and rehabitation.

The emitters can be located inside or outside of the volume requiring remediation. However, it is highly preferred to locate the emitter inside the volume requiring remediation, since then no contaminated air is allowed to leave the volume requiring remediation.

Monitoring and controlling the dew point within the volume requiring remediation is a significant aspect. During the process of decontamination, steps must be taken to avoid condensation. Therefore during the entire decontamination process the atmosphere within the volume requiring remediation must be carefully controlled using space heaters or the HVAC system both to avoid over-humidification and to regulate the temperature of the chlorine dioxide solution fed to the emitter. Failure to control these factors can lead to spot damage as well as a higher use of chlorine dioxide.

As used herein, “CT” equals the time weighted average chlorine dioxide concentration multiplied by the exposure time in hours. In a plot of chlorine dioxide concentration over exposure time in hours, the CT would equal the area under the curve. For example, if the time weighted average chlorine dioxide concentration over a 12 hour exposure period were 750 ppmᵥ, the CT would be 9000 ppmᵥ-hours.

It is an object of this invention to minimize the chlorine dioxide concentration, CT, and relative humidity (RH) as much as possible to ensure in situ remediation of the Chinese wallboard, while avoiding damage to building contents such as electronic equipment (e.g., telephone equipment, computers, copiers, and other electronic office equipment), furnishings, and the like.

Based on past remediation efforts, it has been generally accepted that in order to achieve adequate bacterial kill, chlorine dioxide fumigation of a building requires a minimum relative humidity (RH) of about 65%, with a target ClO₂ concentration and exposure time of 750 ppmᵥ for 12 hours, for a total concentration of 9000 ppmᵥ/hrs (CT). Other researchers have recommended a RH of greater than 70% for ClO₂ concentrations between 125 and 10550 ppmᵥ. Under current EPA guidelines, applications of ClO₂ for building remediation require 75% relative humidity and an exposure of 9000 ppmᵥ/hrs.

An EPA report issued September 2008, entitled Material Demand Studies: Interaction of Chlorine Dioxide Gas with Building Materials, described glove box tests carried out at RH above 75% and a temperature above 25° C. on samples of carpet, painted steel, gypsum wallboard, ceiling tile, wood, and concrete. Concentrations of chlorine dioxide of 1000 ppmᵥ and 2000 ppmᵥ were employed, with a target CT of 12,000 ppmᵥ/hrs. The chlorine dioxide demand varied with the type of building material, but significant operational problems were encountered during the tests, the result of corrosion of electronic components, flow meters, and pumps. Corrosion was also observed on the stainless steel parts within the test chamber.

In accordance with the present invention, chlorine dioxide concentrations are in the range of about 500 ppmᵥ to about 3000 ppmᵥ, and exposure times are about 8 hours to about 12 hours. For sulfate reducing bacteria remediation, a time averaged chlorine dioxide gas concentration of about 9000 CT is effective for killing SRB, mitigating MCI and eliminating allergenic effects.

### Example 3

### Chinese Wallboard Contamination Field Testing

### Introduction

Media reports indicate widespread concern exists among homeowners and apartment dwellers living in structures containing Chinese wallboard that the wallboard gives off gases that can corrode copper pipes, blacken jewelry and silverware, and possibly sicken people.

A study funded by the Florida Department of Health (FDOH) confirmed that Chinese wallboard does indeed have the potential to evolve a number of reduced-sulfur gases under temperature and relative humidity (RH) conditions common in the southeastern US. The FDOH study identified hydrogen sulfide, carbonyl sulfide and carbon disulfide as evolving from Chinese wallboard samples when exposed to elevated RH levels. None of these gases has been shown to evolve from comparable American drywall products at any RH level.

A separate analysis of Chinese wallboard by the US Environmental Protection Agency (USEPA) did not show the presence of any of these three compounds in the Chinese drywall materials themselves. This finding suggests that the gases are formed by some chemical and/or biological activity occurring within the wallboard once it is in place and exposed to high temperature and RH conditions, although a definitive determination has not been made as to the mechanism.

One technology that shows great promise for solving the Chinese wallboard problem is a gaseous chlorine dioxide (ClO₂) fumigation process originally developed by Sabre Technical Services, LLC (Sabre) while assisting USEPA and the US Postal Service (USPS) in devising a technical solution to widespread Bacillus anthracis (i.e. anthrax) contamination present in buildings following the anthrax attacks of 2001. Sabre's ClO₂ fumigation technology was used to eliminate anthrax contamination from the Hart Senate Office Building and USPS Curseen-Morris Processing and Distribution Center (P&DC) in Washington, D.C., the USPS Trenton P&DC in Hamilton Township, N.J. and the former American Media, Inc. Building in Boca Raton, Fla. The size of these ClO₂ fumigation applications ranged from a low of 100,000 cubic feet (ft³) to a high of over 14 million ft³.

Preliminary test work conducted at Sabre's research and development facility in Slingerlands, N.Y. using samples of Chinese wallboard obtained from various affected structures indicated that ClO₂ did indeed hold potential as remedial treatment agent for installed wallboard material. As such, a field technology demonstration project was scheduled at a problem residence in Ft. Myers, Fla. on Jun. 6, 2009 to confirm laboratory observations regarding penetration of ClO₂ in an actual affected structure.

### Project Objectives

Objectives of this field technology demonstration project were to: 1.) document that the ClO₂ fumigation process would result in gas penetration throughout the structure leading to effective elimination of odorous reduced-sulfur compounds; 2.) verify that ClO₂ would not cause unacceptable changes within a treated structure in terms of metal corrosion or material bleaching; and 3.) further investigate the ability of ClO₂ to inactivate sulfate-reducing bacteria (SRBs) present within wallboard material in case it was eventually determined that they played a meaningful role in the reduced-sulfur gas evolution problem.

### Efficacy Sampling Approach

A major complication in determining success of ClO₂ in eliminating reduced-sulfur compounds from an affected structure is the difficulty of measuring and analyzing these gases at the low concentrations they are present at within the structure. Sabre used various surrogate measures to document the efficacy of ClO₂ gas in ridding the test structure of reduced-sulfur compounds.

Gas Penetration—The effects of substrate oxidation occur before effective microbial kill takes place during ClO₂ treatment. A certain minimum “concentration×time” (CT) value must be first accumulated in order to overcome the natural oxidative “demand” of substrate materials prior to achieving microbial kill. This principal forms the basis for decision-making when calculating dosing levels in both liquid and gaseous ClO₂ applications. Therefore, to the extent that pervasive microbial kill can be shown throughout a structure, including inside wall cavities and within substrate materials themselves, it is reasonable to conclude that reduced-sulfur compounds in those locations have also been effectively oxidized.

In order to demonstrate that pervasive microbial kill took place throughout the test structure, and by implication effective oxidation of reduced-sulfur compounds, Sabre's testing approach included two surrogate measures of microbial kill. First, Chinese wallboard has been shown to contain elevated SRB levels compared to conventional wallboard, particularly in the unpainted paper layer. Thus, testing of SRB levels in this layer both pre- and post-treatment provides a good indication of how well ClO₂ gas penetrated into the wallboard and oxidized any reduced-sulfur compounds present in the material. Second, biological indicator (BI) spore strips containing a known titer of Bacillus atrophaeus bacterial spores were deployed inside wall cavities at representative locations throughout the structure. The B. atrophaeus species is widely recognized as being the most difficult to inactivate with ClO₂ gas. Pervasive inactivation of BIs in “hard to reach” areas of the structure (i.e. inside wall cavities) thus indicates that pervasive oxidation of reduced-sulfur compounds also occurred throughout the structure.

Subjective Odor Elimination—Reduced-sulfur compounds odors are extremely noxious and can be detected by the human olfactory (i.e. odor) sense at levels which are at or below the detection limits of sophisticated analytical instruments. As such, the olfactory senses of both Sabre personnel and independent observers were employed both pre- and post-treatment to gauge the effectiveness of ClO₂ in ridding the test structure of reduced-sulfur compound odors.

Elimination of Copper Blackening Effect—Reduced-sulfur compounds have been shown to blacken and corrode copper materials in affected structures over time. Exposure durations in contaminated buildings that result in blackening occurring have been reported as being from one to four weeks under typical environmental conditions. Untarnished copper coupons were placed within the test structure post-treatment and were monitored over time.

### Test Structure

A Courtyard Home with a “Berkshire Floor Plan” located at 5683 Kensington Loop in The Residences at Bell Tower Park in Fort Myers, Fla. was used as the field technology demonstration site. This 2,429 square foot two-story home consists of 3 bedrooms, 3.5 baths, a kitchen, grand room, dining room, laundry room and an attached 2-car garage. This home also has an adjacent 286 square foot guest cabana consisting of 1 bedroom, 1 bathroom and a small kitchen. The main home and guest cabana are connected by a private courtyard with a screen ceiling enclosure, brick foundation and small spa.

The entire structure, including main home, guest cabana and private courtyard was enclosed with impermeable polyethylene sheeting material during the fumigation to prevent release of ClO₂ gas to the surrounding environment.

### Test Methods and Materials

Efficacy of the ClO₂ fumigation process was monitored in several different ways. Key process parameters were monitored throughout the fumigation period to ensure that target treatment conditions were achieved within the affected structure. These process parameters included temperature, RH, ClO₂ concentration and fumigant dose, which is expressed in terms of ClO₂ CT “credits.”

Pre- and post-treatment SRB samples were collected from wallboard material throughout the structure to assess efficacy of the ClO₂ gas in inactivating bacteria present within them, and thus oxidizing any reduced-sulfur compounds. BI spore strips were also placed in representative locations throughout building wall cavities to document that pervasive gas penetration occurred throughout the structure.

Visual and olfactory observations were made by Sabre personnel, as well as by independent parties, on a number of important variables including corrosivity potential of ClO₂ on copper and other metals, bleaching potential of ClO₂ on carpeting and odor presence within the structure both pre- and post-treatment.

Temperature and RH—Temperature and RH conditions within the structure were monitored throughout the fumigation at four representative locations. Each monitored location was deemed to be a potential problem area for controlling temperature and RH conditions based on the home's heating, ventilation and air conditioning (HVAC) system and airflow movement characteristics. Selected monitoring locations were in the 1st floor master suite closet; inside the attic access point in the garage; in the guest cabana kitchen; and inside the attic access point in the 2nd floor suite #2 closet.

The target temperature and RH conditions chosen for the fumigation were a temperature of 80° F.±5° F. and an RH level of 45%±5% at all monitoring locations.

Temperature and RH levels were monitored through use of HOBO® Model U12-011 TEMP/RH Data Loggers manufactured by Onset Computer Corporation. The instrument temperature measuring range is −4 to 158° F. with an accuracy of ±0.63° F. The RH measuring range is 5% to 95% with an accuracy of ±2.5%. Temperature and RH measurements were monitored on a real-time basis and logged at 5-minute intervals throughout the fumigation process.

ClO2 Concentrations and CT Values—ClO₂ concentration levels were monitored throughout the fumigation process at the same four representative locations selected for temperature and RH monitoring. These locations were, again, selected based on knowledge of the home's HVAC systems and airflow movement characteristics.

The target ClO₂ parameters selected for this project were an average concentration of 500 ppmᵥ or more and a CT value not less than 2,000 ppmᵥ nor more than 9,000 ppmᵥ at all monitoring locations. Monitoring of ClO₂ concentrations began shortly after the gas was first introduced into the structure and continued at periodic intervals throughout the fumigation process.

Monitoring was accomplished by means of a sample collection system constructed of one-quarter inch inside diameter high-density polyethylene (HDPE) tubing. The HDPE tubing was run from the four designated monitoring locations to a central sampling manifold located outside the building in a mobile laboratory facility. Samples were collected and analyzed by trained technicians. Air flowed continuously to the sampling manifold so that samples represented existing conditions within the building at the time they were taken. A vacuum pump was placed on the downstream side of the sampling manifold to move air through the system and return it to the structure on a continuous basis throughout the fumigation process.

Samples were collected from the sampling manifold via impingement of two liters of air at a flow rate of 1.0 liter per minute through 15 milliliters of a strongly buffered pH 7 potassium iodide solution (modified US Occupational Safety and Health Administration Method ID126SGX). Once collected, samples were analyzed by colorimetric titration, using a 0.1 normal sodium thiosulfate solution as the titrant (modified American Water Works Association Method 4500-ClO₂-E and modified 2-step version of same).

A fumigation ClO₂ CT dose “clock” was started for each of the four co-located monitoring points when temperature and RH conditions had equilibrated in their desired ranges and gas introduction into the structure had begun. Once started, each CT clock accumulated ClO₂ exposure “credit” until the target dose level had been achieved at each monitoring location, at which time the fumigation was deemed complete.

SRBs—The efficacy of ClO2 gas in eliminating SRBs from Chinese wallboard material was evaluated by collecting samples of unpainted wallboard paper located inside wall cavities of the home prior to, and immediately after, ClO2 exposure. Unpainted wallboard paper from wall cavities was chosen for SRB testing because preliminary laboratory work done at Sabre's Slingerlands, N.Y. laboratory facility had shown SRBs to be concentrated in this media.

Pre-treatment wallboard paper samples were collected by drilling a two-inch circular core at selected wall and ceiling locations. To avoid damaging vapor barriers present within the home, samples were not collected from any bathroom or laundry room locations. Sample locations were selected to be representative wall cavities within the structure most likely to contain conditions conducive to SRB growth. In total, 20 sample locations were selected. Nine were wall cores and eleven were ceiling cores.

The wallboard holes created through SRB sampling were each sealed using a two-inch rubber expansion plug in order to ensure that ClO₂ gas would not penetrate into wall cavities as a consequence of sampling activities.

Post-treatment wallboard paper samples were collected by drilling an identical two-inch circular core approximately one inch away from each of the 20 pre-treatment sample locations.

Following collection, wallboard paper samples were sent to EMLab P&K for independent third party analysis using Method C461—Sulfate Reducing Bacteria Analysis—Presence/Absence.

BI Spore Strips—BI spore strips, each containing an approximate 2.5×103 titer of B. atrophaeus spores, were placed within wall cavities of the structure at the same 20 locations where wallboard samples had been collected, prior to insertion of the 2-inch expansion plugs. The B. atrophaeus species was selected due to its historical use as a biological indicator for ClO2 fumigations

Spore strips are thin cellulose pads that have been impregnated with a defined titer of bacterial spores. Each spore strip is encased in a Tyvek® pouch to allow for effective penetration of fumigant gas yet protect the strip from contamination by external sources. The BIs were obtained from SGM Biotech Inc., 10 Evergreen Drive, Suite E, Bozeman, Mont. (Lot #ACD-113e). All BIs were supplied from the same product batch in order to ensure uniformity in spore titer. Relevant production QA/QC data for the specific lot number have been kept on file for future reference.

All BIs were retrieved promptly following fumigation and sent to Sabre's Slingerlands, N.Y. laboratory facility for analysis. Each spore strip was aseptically placed in a growth media tube containing 15 milliliters of trypticase soy broth (BD Diagnostics product #221823, Lot # 7337460) and incubated at 37 degrees Centigrade. Spore strips were evaluated daily for the presence or absence of indicator organism growth for a total of seven days.

### Visual and Olfactory Observations

The corrosivity potential of ClO₂ on metals and bleaching potential of ClO₂ on household carpeting were evaluated through pre- and post treatment visual observations made throughout the structure.

Corrosivity potential was assessed by observation of typical metal items present within the structure (e.g. screws, door hinges, HVAC system components, etc.). Several pieces of copper pipe were also placed on the Café countertop for the duration of fumigation to verify that ClO₂ would not cause any adverse effects such as corrosion or discoloration. Each piece of copper was “scuffed” clean prior to fumigation to ensure that any changes in the metal due to ClO₂ exposure would be readily recognizable. Photographs were taken of the copper pipe pieces before and after treatment to document visual observations made.

Bleaching potential of ClO₂ was assessed by observation of carpet color and brightness throughout the structure both pre- and post-treatment. A piece of carpeting was also removed from a closet within the structure prior to fumigation and used for direct visual comparison with treated carpet following completion of the process.

Odor levels emanating from within the structure were observed both pre- and post-treatment for the “putrid” characteristic commonly associated with reduced-sulfur gases such as hydrogen sulfide, carbonyl sulfide and carbon disulfide that have been definitively shown by an FDOH study as being released from Chinese wallboard.

### Quality Control

BI Spore Strips—Positive control BIs were submitted to the Sabre laboratory for viability testing along with the fumigated BIs in a ratio of approximately one positive control sample for every 10 treated samples, for a total of two positive controls. Positive controls are untreated (i.e., not fumigated) BIs of identical composition that are submitted to the laboratory along with the exposed BIs. Positive controls provide evidence of BI product quality as well as evidence that appropriate conditions for growth of the surrogate test organism were achieved. The positive control samples were handled, packaged and shipped in the same manner as the actual samples from the building, except that the positive controls were not subjected to the fumigant gas.

### Results

Temperature and RH—Raw temperature and RH data were exported from the HOBO® data loggers into a Microsoft Corporation Excel® spreadsheet for purposes of calculating mean temperature and RH levels for each monitoring location. These mean temperature and RH values (±one standard deviation) are shown in Table 1.

TABLE 1
Temperature & RH Data Summary

| | | Actual | | | |
|---|---|---|---|---|---|
| | | Line 101 | Line 102 | Line 103 | Line 104 |
| | Tar- | Master Suite | Garage Attic | 2nd Floor | Guest |
| | get | Closet | Access | Attic Access | Cabana |
| Temp | 80 | 76.5 (±1.9) | 81.1 (±5.8) | 82.6 (±5.2) | 76.9 (±2.4) |
| (° F.): | | | | | |
| RH | 45 | 47.8 (±1.0) | 48.2 (±1.9) | 45.1 (±2.6) | 51.7 (±0.8) |
| (%): | | | | | |

Monitoring data showed that temperature and RH were maintained close to target levels throughout the fumigation. The slightly elevated RH level observed in the Guest Cabana (51.7%) was believed to be the result of water present in the courtyard spa.

ClO2 Concentrations and CT Values—Raw sample collection and analytical data were entered into a Microsoft Corporation Excel® spreadsheet for purposes of calculating mean ClO2 concentrations and accumulated CT values for each monitoring location. These mean ClO2 concentration and CT values (±one standard deviation) are shown in Table 2.

TABLE 2
ClO₂ & CT Data Summary

| | | Actual | | | |
|---|---|---|---|---|---|
| | | Line 101 | Line 102 | Line 103 | |
| | | Master | Garage Attic | 2nd Floor | Line 104 |
| | Target | Suite Closet | Access | Attic Access | Guest Cabana |
| Time (hours): | 4+ | 13 | 13 | 13 | 13 |
| ClO₂ (ppmᵥ): | 500+ | 695 (±298) | 685 (±267) | 475 (±218) | 825 (±340) |
| CT (ppmᵥ-hours): | 2000-9000 | 8090 | 8061 | 5336 | 9727 |

Monitoring data showed that ClO₂ concentrations and CT values were maintained within target ranges established for the fumigation. A mean ClO₂ concentration slightly less than 500 ppmᵥ was maintained at the 2^{nd} floor attic access point, however a corresponding CT greatly in excess of the 2,000 ppmᵥ-hour minimum was also achieved at this location.

SRBs SRB growth test results for the 20 unpainted wallboard paper samples collected from within wall cavities before and after fumigation and sent to EMLab P&K are summarized in Table 3.

TABLE 3
SRB Summary Data

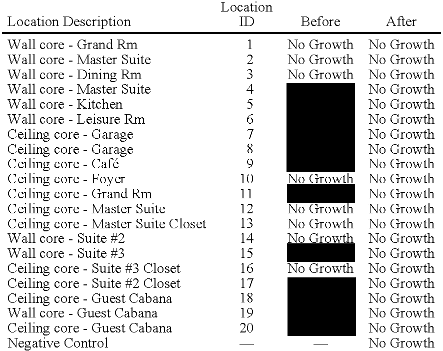

The SRB growth data indicated a widespread presence of SRBs within the unpainted wallboard paper prior to fumigation. Twelve of 20 sample locations were found to be positive for SRBs prior to ClO₂ treatment. Following treatment, all 20 locations were determined to be negative for SRB growth.

BI Spore Strips—Viability test results for the 20 BI spore strips placed within wall cavities of the structure during fumigation are shown in Table 4.

TABLE 4
Spore Strip Summary Data

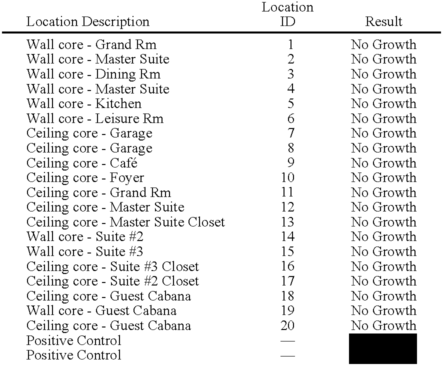

The BI test results verified that pervasive, efficacious ClO₂ gas penetration occurred throughout the structure, including inside wall cavities, during fumigation. Each of 20 log 10³ Bacillus atrophaeus spore strips placed in very challenging locations within the wall cavities were found to be negative for surrogate test organism growth following ClO₂ treatment.

Both positive control BI spore strip samples were found to be positive for indicator organism growth, thereby indicating that BI product quality was good and that appropriate conditions for growth of the surrogate test organism were achieved in the laboratory.

### Visual and Olfactory Observations

Observations made of common metal items present within the structure following fumigation indicated no corrosive effect was visible from exposure to the ClO₂ gas. Similarly, no changes were observed in the pieces of copper pipe placed on the Café countertop, with the minor exception that some pieces appeared to have a “gold-like” tint following treatment.

Observations made of carpet color and brightness throughout the structure following fumigation indicated no meaningful bleaching effect had occurred from exposure to the ClO₂ gas. A direct side-by-side comparison of treated carpet with a piece of untreated carpet removed from the structure prior to fumigation confirmed this finding. It should be noted that each dye lot and color of carpet behaves differently and needs to be individually evaluated.

Putrid odors characteristic of reduced-sulfur compounds known to evolve from Chinese wallboard were readily apparent to both Sabre personnel and independent observers throughout the structure prior to fumigation, and were particularly strong in the garage and cabana areas. Following fumigation, a faint “swimming pool like” scent was present in the structure from use of ClO₂ gas, but the reduced-sulfur gas odors appeared to have been completely eliminated.

### CONCLUSIONS

All process parameter targets, including temperature, RH, ClO₂ concentration and CT values, were achieved during this field technology demonstration project and all objectives were satisfied.

The ClO₂ fumigation process was shown capable of inactivating SRBs present within wallboard material, as well as BI spore strips embedded within wall cavities, thereby demonstrating the ability of ClO₂ gas to completely permeate an affected structure and oxidize reduced sulfur compounds at the CT values employed. In addition, it was demonstrated that ClO₂ would not cause unacceptable changes within a treated structure in terms of metal corrosion or material bleaching.

The present invention is not to be limited in scope by the specific embodiments described herein, but by the appended claims. The described embodiments are intended as illustrations of individual aspects of the invention, and functionally equivalent methods and components are within the scope of the invention. Indeed, various modifications of the invention, in addition to those shown and described herein will become apparent to those skilled in the art from the foregoing description and accompanying drawing. Such modifications are intended to fall within the scope of the appended claims.

### LIST OF ACRONYMS

### BI Biological Indicator

### CFM Cubic Feet Per Minute

### ClO₂ Chlorine Dioxide

### CT Concentration×Time

### DFU Dry Filter Unit

### F Fahrenheit

### FDOH Florida Department of Health

### HDPE High Density Polyethylene

### HVAC Heating, Ventilation and Air Conditioning

### P&DC Processing and Distribution Center

ppmᵥ Parts Per Million by Volume

### RH Relative Humidity

### Sabre Sabre Technical Services, LLC

### SRBs Sulfate-Reducing Bacteria

### USEPA US Environmental Protection Agency

USPS US Postal Service

1. A method for eliminating out-gassing of reduced sulfur and sulfur gases from gypsum wallboard, said method comprising: exposing at least one gypsum wallboard surface to chlorine dioxide gas.

2. The method of claim 1 wherein said gypsum wallboard is disposed within an enclosed volume, and said chlorine gas is introduced into said enclosed volume at a CT value of about 150 ppmᵥ-hrs to about 50,000 ppmᵥ-hrs.

3. The method of claim 2 wherein said CT value is about 1000 ppmᵥ-hrs to about 29,000 ppmᵥ-hrs.

4. The method of claim 2 wherein said enclosed volume is at a temperature of about 50° C. to about 90° C.

5. A method for in situ remediation of gypsum wallboard comprising exposing at least one gypsum wallboard surface within an enclosed volume to chlorine dioxide gas at a CT value of about 150 ppmᵥ-hrs to about 50,000 ppmᵥ-hrs.

6. The method of claim 5 wherein said CT value is about 1000 ppmᵥ-hrs to about 29,000 ppmᵥ-hrs.

7. The method of claim 6 wherein said CT value is about 9000 ppmᵥ-hrs.

8. The method of claim 5 wherein said enclosed volume is at a temperature of about 10° C. (50° F.) to about 32° C. (90° F.).

9. A method for eliminating contaminants in gypsum wallboard that cause noxious sulfide odors, said method comprising:
exposing to chlorine dioxide gas at least one gypsum wallboard surface within an enclosed volume;
wherein said chlorine dioxide gas is introduced into said enclosed volume under conditions of chlorine dioxide gas concentration and contact time, and humidity effective to eliminate the noxious odor-causing contaminants in the gypsum wallboard.

10. The method of claim 9 wherein said contaminants within said enclosed volume comprise bacteria.

11. The method of claim 10 wherein said bacteria comprise sulfate reducing or thiophilic bacteria.

12. The method of claim 9 wherein said enclosed volume further comprises objects selected from the group consisting of metallic objects, non-metallic objects, and combinations thereof.

13. The method of claim 12 wherein said metallic objects are formed from metals selected from the group consisting of steel, aluminum, iron, copper, chromium, lead, and combinations thereof.

14. The method of claim 12 wherein said non-metallic objects are formed from materials selected from the group consisting of wood, brick, stone, cinder concrete, ceramic tile, ceiling tile, carpet, woven fabric, and combinations thereof.

15. The method of claim 9 wherein said chlorine dioxide gas is introduced into the enclosed volume at a CT value of about 150 ppmᵥ-hrs to about 50,000 ppmᵥ-hrs.

16. The method of claim 15 wherein said CT value is about 1000 ppmᵥ-hrs to about 29,000 ppmᵥ-hrs.

17. The method of claim 16 wherein said CT value is about 9000 ppmᵥ-hrs.

18. The method of claim 9 wherein said chlorine dioxide gas is introduced into the enclosed volume at a concentration of about 25 ppmᵥ to about 10,000 ppmᵥ.

19. The method of claim 18 wherein said chlorine dioxide gas is at a concentration of about 500 ppmᵥ to about 5,000 ppmᵥ.

20. The method of claim 9 wherein said enclosed volume is at a temperature of about 10° C. (50° F.) to about 32° C. (90° F.).

21. The method of claim 20 wherein said enclosed volume is at a temperature of about 18° C. (65° F.) to about 29° C. (85° F.).
